(19) United States
(12) Patent Application Publication
(10) Pub. No.: US 2021/0170106 A1
(43) Pub. Date: Jun. 10, 2021

(54) ASSISTIVE DEVICE FOR USE WITH A SYRINGE

(71) Applicant: Teva Pharmaceuticals International GmbH, Basel (CH)

(72) Inventors: Christopher Louis BELISLE, Somerset, WI (US); Daniel Casey JOHNSON, Minneapolis, MN (US)

(21) Appl. No.: 17/271,526

(22) PCT Filed: Aug. 14, 2019
(86) PCT No.: PCT/EP2019/071861

§ 371 Date: Feb. 25, 2021

(30) Foreign Application Priority Data
Aug. 28, 2018 (EP) 18191283.3

(51) Int. Cl. A61M 5/315; A61M 5/32

(52) U.S. Cl. CPC A61M 5/3158; A61M 5/3202; A61M 5/31591; A61M 5/31511

(57) ABSTRACT

An assistive device for use with a syringe comprises a housing for accommodating the syringe, a dispense member to be moved in a distal direction for dispensing a medicament from the syringe and a needle guard for covering a needle of the syringe before operation of the device. The dispense member comprises a first locking device and the housing comprises a second locking device for locking the dispense member to the housing when the needle guard in is a distal position, such that a movement of the dispense member in the distal direction is prevented.

### FIELD OF THE INVENTION

The present disclose relates to an assistive device for use with a syringe, in particular a syringe prefilled with a medicament. The assistive device serves to improve the handling of the syringe. The device may conceal the syringe and, in particular, the needle, in order to help users not being confident with handling a needle. The assistive device may simplify placing the dispensing end of the device correctly on the skin and administering the medicament.

### BACKGROUND

U.S. Pat. No. 9,233,213 B2 discloses a palm-activated drug delivery device for dispensing medicament from a pre-filled barrel.

### BRIEF SUMMARY OF THE INVENTION

It is an object of the present disclosure to provide an assistive device for use with a syringe, the assistive device having improved properties. Specific embodiments may provide increased user comfort, increased user safety, reduced error-proneness and/or reduced manufacturing costs.

A first aspect of the present disclosure relates to an assistive device for use with a syringe. The syringe may be a traditional syringe prefilled with a medicament. The syringe may comprise a needle and a barrel containing the medicament. The syringe may be usable as a stand-alone syringe, i.e., without the assistive device. Such a syringe typically comprises a plunger being partially accommodated in the barrel and protruding out of the barrel. The plunger is movable in a distal direction for pressing the medicament out of the barrel. A flange may be located at a proximal end of the barrel for providing a counter bearing for a user's fingers when using the syringe without the assistive device.

In general, the term “distal” or “distal end” refers to that portion or end of the device or a component thereof that is closest to a dispensing end of the device. The dispensing end is the part of the device through which the medicament is dispensed from the device. The term “proximal” or “proximal end” refers to that portion or end of the device or a component thereof that is furthest away from the dispensing end of the device. The term “distal direction” or “distally” refers to the direction defined by a direction from the proximal end to the distal end of the device or a component. The term “proximal direction” or “proximally” refers to the opposite direction, i.e., is defined by a direction from the distal end to the proximal end.

The device comprises a housing for accommodating the syringe. The housing may be an outer housing which is held by a user when operating the device. The housing may comprise a distal housing and a proximal housing. The distal and proximal housing may be releasably or unreleasably coupled to each other. In specific embodiments, the housings may be coupled releasably to each other before operation of the device and may be unreleasably locked to each other after operation of the device.

The device comprises a dispense member to be moved in a distal direction for dispensing the medicament from the syringe. The movement of the dispense member may be accomplished by manually applying a force on a proximal surface of the dispense member.

The dispense member may have an initial, proximal position before starting a dose dispense operation. During dispensing a dose, the dispense member may be moved from the proximal position in a distal direction until a stop is reached and the dispense member cannot be moved further in the distal direction. In specific embodiments, this position of the dispense member may be the final, distal position.

The dispense member may directly act on the syringe plunger. For this aim, the dispense member may comprise a post extending from a proximal end of the dispense member in a distal direction and acting on the proximal surface of the plunger for pushing the plunger forwards. The amount of axial movement of the dispense member and the plunger during a dispense operation may be identical.

The device comprises a needle guard for covering a needle of the syringe before operation of the device. In this state, the needle guard may be in an initial, distal position. For initiating a dispense operation, the needle guard is movable from the initial distal position in a proximal direction to expose the needle such that the needle protrudes out of a distal end of the device. This is achieved by a user placing the dispensing end of the device on his/her skin and pressing the device towards the skin. This action may push the needle guard in a proximal direction relative to the housing, thereby exposing the needle, which pierces the skin. When the needle guard has been fully pushed in the proximal direction it reaches a stop and cannot move further in the proximal direction. In specific embodiments, this position may be the final proximal position.

The assistive device may have different states. In particular, the device may have a locked state, which is present before a dispense operation has been carried out. In the locked state, the dispense member is locked such that a movement of the dispense member in a distal direction is disabled. Thereby, inadvertent dispense of the medicament can be prevented. This may include cases where the device has not yet been positioned at an injection side and the needle has not been exposed.

In particular, the dispense member may comprise a first locking device and the housing may comprise a second locking device for locking the dispense member to the housing. The locking devices may interact with each other to lock the dispense member to the housing prior to a proximal movement of the needle guard.

The locking may be achieved without any interaction with further components such as the needle guard. In particular, the needle guard may be not in contact with the dispense member in the locked state. As an example, the needle guard may be positioned at a distance from the dispense member. In particular, the proximal end of the needle guard may be located at a more distal position than the distal end of the dispense member.

In particular, the housing may comprise a proximal housing and a distal housing to be connected to each other for assembly of the device. The proximal housing may be part of a proximal assembly that also comprises the dispense member. The distal housing may be part of a distal assembly that also comprises the needle guard. For assembly of the device, the syringe may be placed first in the distal assembly and then the proximal assembly may be coupled to the distal assembly. The proximal housing may comprise the second locking device. In this case, the dispense member may be locked with the proximal housing also prior to assembly of the device. Accordingly, the locking may be already present in a pre-assembled state of the device.

Thereby, the assembly of the device may be simplified, because the dispense member is not a movable part in the proximal assembly. In specific embodiments, neither the proximal assembly nor the distal assembly comprises any parts movable to the respective housing before assembly of the proximal housing with the distal housing.

The device may comprise a release device for unlocking the locking devices and, thereby, the dispense member, when the needle guard has moved in a proximal direction. In particular, the needle guard may have a most distal position and a most proximal position relative to the housing and the unlocking may occur when the needle guard is in or near its most proximal position.

In specific embodiments, the needle guard may comprise the release device for unlocking the dispense member. When the dispense member is in its unlocked state, the first locking device may no longer interact with the second locking device. In this state, the dispense member is movable in the distal direction.

The dispense member may comprise a flexible component providing the first locking device. In particular, the flexible component may have the shape of a flexible arm. The flexible arm may extend from a proximal part of the dispense member in a distal direction.

In the assembled device, the flexible arm may be permanently biased away from its relaxed state. In particular, a tip of the flexible arm may be flexed inwards from its relaxed state by abutment with the housing or the needle guard, depending on the state of the device. Accordingly, the tip may exert an outwardly directed counterforce on the housing or the needle guard, respectively. The flexible arm, in particular the tip of the flexible arm may interact with the housing to lock the dispense member as long as the needle guard is in its distal position.

The interaction of the first and second locking devices may be an abutment of the first locking device on the second locking device, in particular an abutment of a distal face of the first locking device on a proximal face of the second locking device. The second locking device may comprise a protrusion. The protrusion may be an inwardly directed protrusion at an inner surface of the housing. The protrusion may have the shape of a ledge.

The device may comprise several first locking devices and several second locking devices. As an example, the dispense member may comprise two flexible arms and the housing may comprise two protrusions located at opposite sides of the device.

The release device may interact with the first locking device of the dispense member, in particular move the first locking device out of interaction with the second locking device. In particular, the release device may push the first locking device inwardly. This may lead to the first locking device being not located above the second locking device anymore such that a movement of the dispense member is not blocked by an abutment on a proximal face of the second locking device. The release device may be provided by a rigid, i.e. non-flexible part. The release device may have a slot to receive the second locking device when moving in a proximal direction.

As an example, the needle guard may comprise a rack. The rack may extend in the proximal direction. The release device may be provided by the rack. In particular, a tip portion of the rack may interact with the first locking device. The needle guard may comprise several racks. As an example, the needle guard may comprise two racks located on opposite sides of the device.

For unlocking the dispense member, the release device may slide between the housing and the first locking device when the needle guard moves in a proximal direction. The first locking device may have a surface for interaction with a surface of the release device. Both surfaces may be beveled to facilitate the interaction. In addition to that, the first locking device may have a protruding portion serving as a spacer to provide a gap between the surface for interaction with the release device and the inner wall of the housing. The spacing facilitates the sliding of the release device between the housing and the surface of the dispense member.

When the dispense member and the housing are unlocked from each other, the device is in an operational state ready to dispense. For dispensing the medicament, the dispense member can now be pushed in a distal direction.

In specific embodiments of the device, the dispense member may be retained in its position during a dispense operation, when the dispense member is inadvertently released. In this regard, during a dispense operation means that the dispense member has not arrived at a final distal position but is still movable distally. As an example, flexible arms of the dispense member may be pretensioned during a dispense operation such that they exert a counterforce on a part of the device, which holds the dispense member in its position. In particular, the flexible arms may slide along the needle guard during a distal movement of the dispense member.

In specific embodiments, the device is a single-use device such that only a single dispense operation is enabled. The single dispense operation includes a movement of the dispense member in a distal direction until a stop is reached. The device may have a locked-out state, in which a further dose dispense operation is disabled.

In an embodiment, the device may be configured such that when the needle guard is in its proximal position and the dispense member is in its distal position, the needle guard interacts with the dispense member such that the dispense member and/or the needle guard is held in its axial position. In particular, the positions of the needle guard and the dispense member at the end of a dose dispense operation may be maintained.

As an example, the needle guard may comprise a first lock-out device and the dispense member may comprise a second lock-out device.

The lock-out devices may engage each other at or near the end of a dose dispense operation. As an example, the first lock-out device may comprise an inwardly directed protrusion. The second lock-out device may comprise an opening for engagement with the protrusion, or vice versa. In particular, a flexible arm of the dispense member may provide the second lock-out device. Accordingly, the flexible arm may both provide the locking device for locking the dispense member prior to a dispense operation and the lock-out device for locking the dispense member after a dispense operation. The locking device and the lock-out device may overlap with each other.

As a further example, lock-out may be provided by friction between the needle guard and the dispense member. In particular, friction may be present between a flexible arm of the dispense member acting on the needle guard.

In the locked state, the dispense member may remain in its most distal position. Furthermore, the needle guard may remain in its retracted position such that the needle remains exposed. In other words, the needle may be permanently visible after dispense of the medication and release from a user's skin. This may clearly signal to a user that the device has been used and should be discarded.

In addition to that, in the locked-out state also a re-use of the syringe may be prevented. In particular, a disassembly of the device may be prevented when the dispense member is locked to the needle guard. Accordingly, a disconnection of the proximal housing from the distal housing may be disabled after dispense of a medication. Also here, locking means that relative movements between the parts, in particular relative axial movements are prevented. Thereby, safety may be increased.

In specific embodiments, the device does not comprise a longitudinally acting spring. As an example, the device is free from a longitudinally acting spring being pretensioned to move the needle guard back or the dispense member in its distal position after use. Among others, this reduces the production costs of the device.

According to a second aspect of the present invention, an assistive device for use with a syringe comprises a housing for accommodating the syringe, a dispense member to be moved in a distal direction for dispensing a medicament from the syringe and a needle guard having a distal position for covering a needle of the syringe before operation of the device, wherein the needle guard is moveable from the distal position to a proximal position to expose the needle. The assistive device is configured such that when the needle guard is in its proximal position and the dispense member is in its distal position, the needle guard interacts with the dispense member such that the dispense member and/or the needle guard is held in its axial position.

The device according to the second aspect may comprise any structural and functional features described in the foregoing in relation to the assistive device of the first aspect. However, the device according to the second aspect is also independent from the subject matters of first and second locking devices.

As an example, the needle guard may comprise a first lock-out device and the dispense member may comprise a second lock-out device engaging each other for locking the needle guard to the dispense member. The second lock-out device may comprise an opening for engagement with the protrusion, or vice versa. This locking may occur at the end of a dose dispense operation.

As a further example locking may be achieved by a frictional force between the dispense member and the needle guard. In particular, a flexible arm of the dispense member may exert a resilient force on the needle guard.

According to a further aspect, a medication delivery kit is disclosed, the medication delivery kit comprising the assistive device and a prefilled syringe accommodated in the assistive device. In particular, the syringe may be filled with a pharmaceutical preparation comprising fremanezumab, an anti-calcitonin gene-related peptide (anti-CGRP) monoclonal antibody for the preventive treatment of migraine.

The medication delivery kit may comprise any structural and functional feature described in the foregoing in relation to the assistive devices and the syringe. The assistive device and prefilled syringe may be packaged or otherwise distributed together. In the assembled state of the assistive device and prefilled syringe, the medication delivery kit is also denoted as medication delivery device.

### BRIEF DESCRIPTION OF THE DRAWINGS/FIGURES

Features which are described herein above and below in conjunction with different aspects or embodiments, may also apply for other aspects and embodiments. Further features and advantageous embodiments of the subject-matter of the disclosure will become apparent from the following description of the exemplary embodiment in conjunction with the figures, in which:

FIG. 1 shows an embodiment of an assistive device in a side view.

FIG. 2 shows the assistive device of FIG. 1 in a pre-assembled state and a syringe to be used with the device.

FIG. 3 shows an exploded view of the assistive device of FIG. 1 and the syringe.

FIGS. 4A to 4C show a proximal assembly of the device of FIG. 1 in a side view, a top view and a longitudinal-sectional view, respectively.

FIGS. 5A to 5C show a dispense member of the assistive device of FIG. 1 in a side view, a rotated side view and a longitudinal-sectional view, respectively.

FIGS. 6A to 6E show a distal housing of the assistive device of FIG. 1 in a perspective view, a side view, a rotated side view, a top view and a longitudinal-sectional view, respectively.

FIGS. 7A to 7E show a needle guard of the assistive device of FIG. 1 in a perspective view, a side view, a rotated side view, a top view and a longitudinal-sectional view, respectively.

FIGS. 8A to 8D show a protective cap of the assistive device of FIG. 1 in a perspective view, a side view, a top view and a longitudinal-sectional view, respectively.

FIGS. 9A to 9C show the assistive device of FIG. 1 comprising a syringe in different states in longitudinal-sectional views, wherein:

FIG. 9A shows the assistive device in a locked state of the dispense member prior to use.

FIG. 9B shows the assistive device in an unlocked state of the dispense member, ready to dispense.

FIG. 9C shows the assistive device in a locked-out state after usage.

Like elements, elements of the same kind and identically acting elements may be provided with the same reference numerals in the figures.

### DETAILED DESCRIPTION

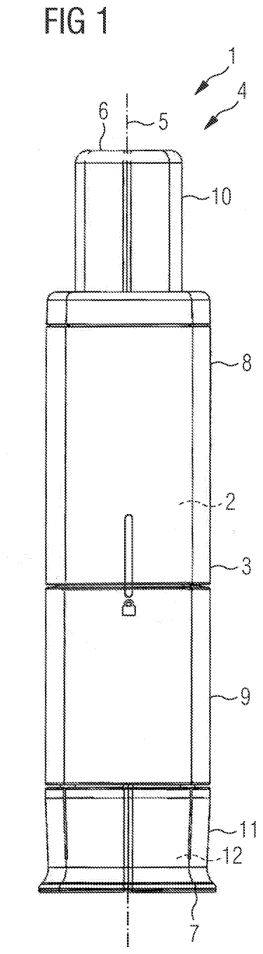

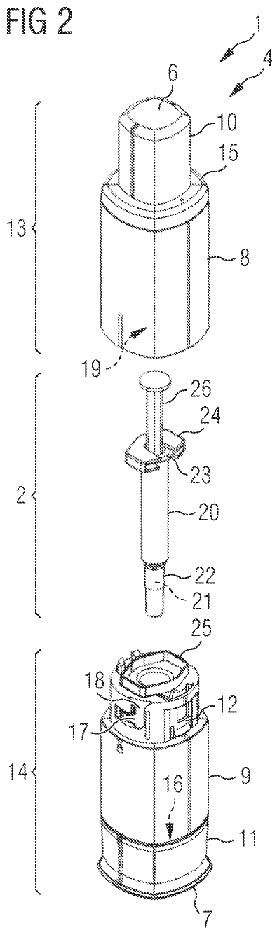

FIG. 1 shows an assistive device 1 for use with a syringe 2. The syringe 2 is accommodated in a housing 3 of the device 1. The syringe 2 is concealed by the housing 3 and is indicated with a dashed reference line. The syringe 2 is explicitly shown in FIG. 2, for example. The housing 3 may have a cross section area in the range of about 4 cm2 to about 16 cm2, for example.

The assistive device 1 assists a user in holding and operating the syringe 2 for administering a medicament. Accordingly, FIG. 1 also shows a medication delivery device 4 comprising the assistive device 1 and the syringe 2.

Embodiments of the device 1 provide a pre-use locking mechanism such that an inadvertent dispense operation is prevented. Additionally or alternatively, embodiments of the device 1 provide a lock-out mechanism such that a re-use of the device 1 after use is disabled. In particular, the device 1 may be a single-shot device, allowing only a single dispense operation. Such devices may be also called single-use devices.

The assistive device 1 has a centerline 5, a proximal end 6 and a distal end 7. The housing 3 accommodates the syringe 2 and is held by a user when operating the device 1. The syringe 2 is fixed within the housing 3 such that any relative movements between a main part of the syringe 2 and the housing 3 are disabled. The housing 3 comprises a proximal housing 8 and a distal housing 9. In the depicted assembled state of the device 1, the distal housing 9 is coupled to the proximal housing 8, for example by a bayonet coupling.

The device 1 comprises a dispense member 10 to be operated by a user for dispensing the medicament. In particular, the dispense member 10 is moved in a distal direction, i.e. in a direction defined by a connecting line from the proximal end 6 to the distal end 7. The movement of the dispense member 10 is accomplished by applying a force, e.g. by the thumb, on a dispense surface of the dispense member 10, which may correspond to the proximal end 6 of the device 1.

The dispense member 10 is shown in its most proximal position, i.e., a state in which the dispense member 10 protrudes distinctly out of the housing 3. This indicates to a user that the device has not be used in a dispense operation.

The device 1 is operated manually by pushing the dispense member 10 in a distal direction. The dispense member 10 may be pushed into its most distal position until a stop is reached. The movement from the most proximal position to the most distal position corresponds to a full dose dispense operation. The force required for injection may be entirely delivered by a user without support by help mechanisms such as a spring. Also the process of penetration of the needle into the skin may be carried out by direct user force.

Accordingly, the device 1 may be a fully manually operated device and not an automatic or a semi-automatic device. In other embodiments the device 1 may be an automatic or a semi-automatic device. As an example, the device 1 may comprises a spring for providing the dispensing force.

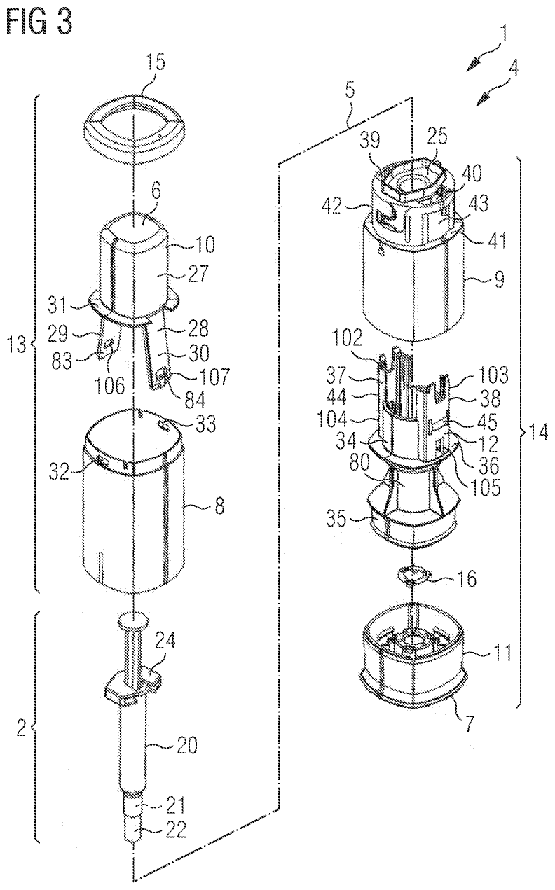

At its distal end 7, the device 1 comprises a protective cap 11, adjacent to the distal housing 9. The protective cap 11 may be coupled to a needle guard 12. In the depicted state, the needle guard 12 is concealed by the protective cap 11 and the housing 3 and is indicated with a dashed reference line. The needle guard 12 is explicitly shown in FIG. 3, for example.

As long as the protective cap 11 is attached, a proximal movement of the needle guard 12 is disabled. In this state, the needle guard 12 is in its most distal position. A proximal movement is a movement in a direction defined by a connecting line from the distal end 7 to the proximal end 6. In particular, a proximal movement is prevented by abutment of a proximal end of the protective cap 11 on a distal end of the distal housing 9.

In the depicted state of the device 1, the syringe 2 is fully concealed. Moreover, the dispense member 10 is locked such that a movement of the dispense member 10 relative to the housing 3 is prevented.

FIG. 2 shows the assistive device 1 of FIG. 1 in a pre-assembled state and a syringe 2 to be used with the device 1. The device 1 and the syringe 1 may be packaged and distributed separately to the user. In another embodiment, the assistive device 1 and the syringe 3 may be provided as a kit such that they are packaged or otherwise distributed together. In both cases, the assistive device 1 may be not yet assembled to each other when being distributed. In other embodiments, the assistive device 1 and the syringe 2 may be in an assembled state when being distributed.

The device 1 may be distributed as two pre-assembled parts, a proximal assembly 13 and a distal assembly 14, not yet attached to each other.

The syringe 2 may be a traditional syringe prefilled with a therapeutic agent. The wordings “medicament” and “therapeutic agent” are used as synonyms in this disclosure.

For assembling the device 1 and the syringe 2, the syringe 2 is in a first step accommodated in the distal assembly 14 and, thereafter, the proximal assembly 13 is placed on top over the syringe 2 and aligned with the distal assembly 14. The proximal assembly 13 is then coupled to the distal assembly 14, via a bayonet coupling, for example.

The proximal assembly 13 comprises the proximal housing 8, the dispense member 10 and a fixation part 15. The fixation part 15 is coupled to the proximal housing 8 to prevent a disassembly of the proximal housing 8 and the dispense member 10.

The distal assembly 14 comprises the distal housing 9, the protective cap 11 and the needle guard 12. The distal assembly 14 further comprises a needle cap grip 16 which is concealed by the distal housing 9 and is indicated with a dashed reference line. The needle cap grip 16 is explicitly shown in FIG. 3, for example.

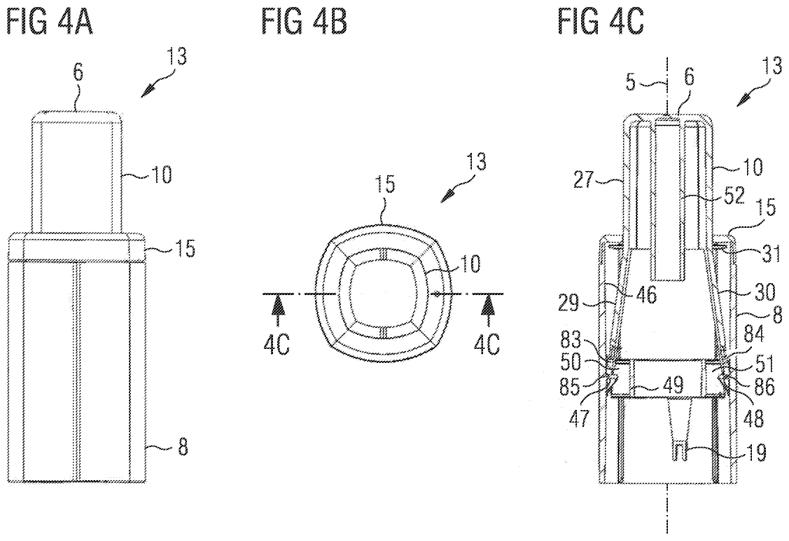

The distal housing 9 comprises a coupling portion 17 comprising a guiding groove 18 for guiding a coupling protrusion 19 of the proximal housing 8 for coupling the proximal assembly 13 to the distal assembly 14. The coupling protrusion 19 is located at an inner surface of the proximal housing 8 and is indicated by a dashed reference line. The coupling protrusion 19 is explicitly shown in FIG. 4C, for example.

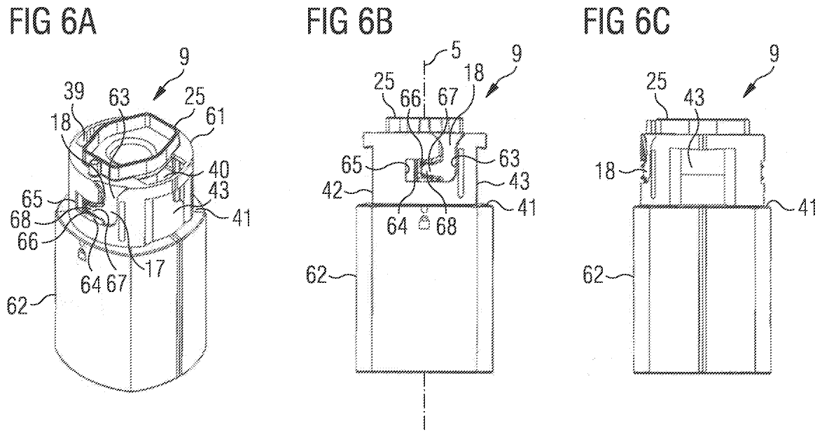

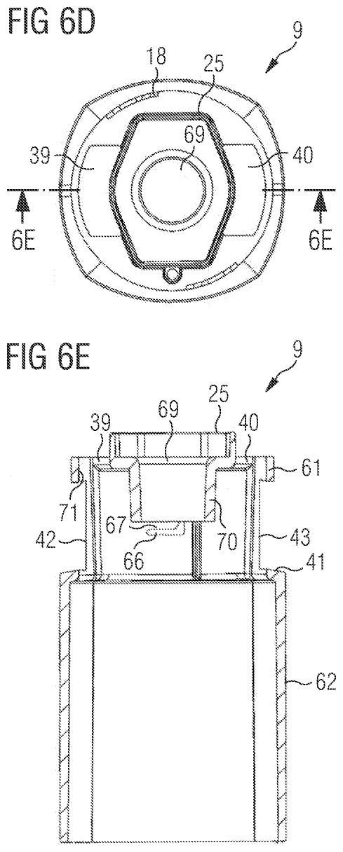

The shape of the guiding groove 17 defines the relative movement of the proximal and distal housings 8, 9 during assembly, corresponding to the relative movement of the proximal and distal assemblies 13, 14. In particular, the guiding groove 17 defines a relative axial movement followed by a relative rotational movement of the housing parts 8, 9. A specific embodiment of the coupling portion 17 is described in connection with FIGS. 6A and 6B.

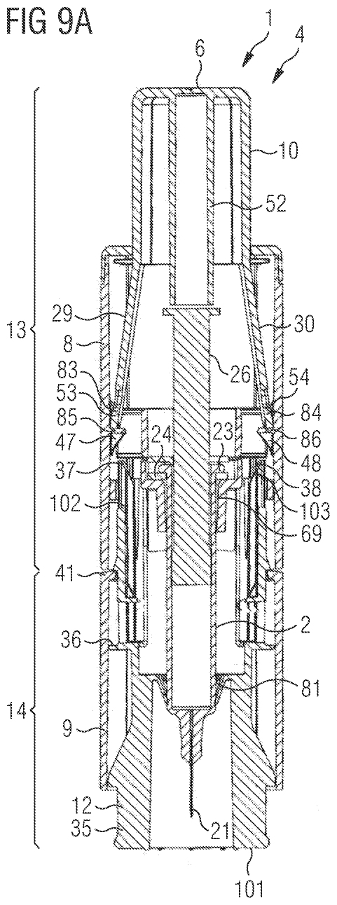

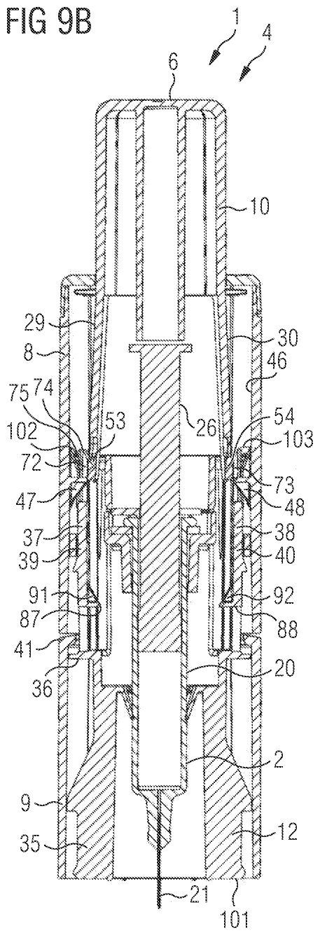

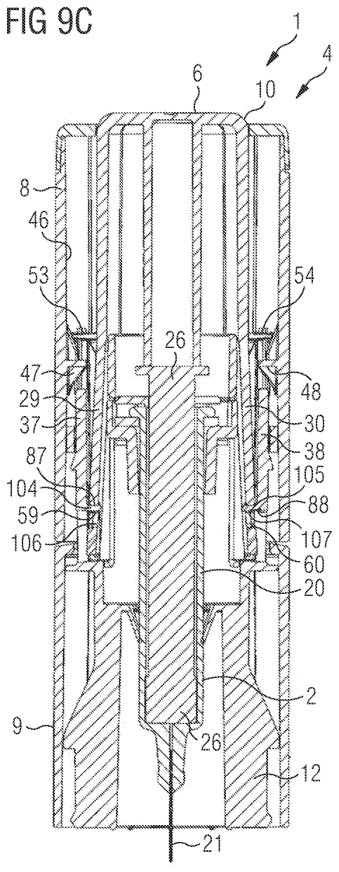

The syringe 2 is also usable as a stand-alone syringe, i.e., without the assistive device 1. The syringe 2 comprises a barrel 20 containing the medicament. The syringe 2 comprises a needle 21 protected by a needle cap 22. The position of the needle 21 is indicated by a dashed line. The needle 21 is explicitly shown in FIG. 9A, for example.

A flange 23 is located at a proximal end of the barrel 20. The flange 23 provides a counter bearing for a user's fingers when using the syringe 2 without the assistive device 1. A fitting 24 is connected to the flange 23, adding surface area and thickness to the flange 23. The fitting 24 may be removable from the flange 23.

When the syringe 2 is positioned in the distal assembly 14, the fitting 24 and flange 23 are accommodated in a cradle 25 provided by the distal housing 9. The outer dimensions of the fitting 24 are matched with the inner dimensions of the cradle 25 such that the fitting 24 accurately fits in the cradle 25. Accordingly, the fitting 24 adapts the dimensions of the flange 23 to the dimensions of the cradle 25. Thereby, the position of the syringe 2 in the distal housing 9 is defined and a rotational movement of the syringe 2 relative to the distal housing 9 is disabled.

The syringe 2 comprises a plunger 26 being partially accommodated in the barrel 20 and protruding out of a proximal end of the barrel 20. When the plunger 26 is moved in a distal direction, the medicament is pressed out of the barrel 20 through the needle 21. A distal end of the plunger 26 may be in direct contact with the medicament or in contact with a piston acting on the medicament. The plunger 26 may be a part belonging intimately to the syringe 2 such that the plunger 26 is unremovably retained in the syringe barrel 20 when distributed to users. Unremovably means that a removal of the plunger is not possible or only possible with great difficulty.

FIG. 3 shows an exploded view of the assistive device 1 and a view of the syringe 2.

Regarding the components of the proximal assembly 13, the dispense member 10 comprises a proximal portion 27 providing a user interface and a distal portion 28 accommodated in the proximal housing 8 in an assembled state of the proximal assembly 13. The distal portion 28 comprises two flexible arms 29, 30.

The flexible arms 29, 30 help to define the axial position of the dispense member 10 in the proximal assembly 13 in specific states of the device 1. In specific embodiments, this may include locking the dispense member 10 in a specific position. The specific states may include one or more of a pre-assembled state, an operational state ready to pierce, an operational state ready to dispense and a state after usage. In particular, the flexible arms 29, 30 may provide first locking devices 83, 84 for locking the dispense member 10 prior to use and second lock-out devices 106, 107 for locking the needle guard 12 after usage. The locking devices 83, 84 and lock-out devices 106, 107 may overlap with each other. The mechanism of the definition and locking of the axial position is described in detail in connection with the following Figures, e.g. in connection with FIGS. 9A to 9C.

The dispense member 10 comprises a flange 31 located between the proximal portion 27 and the distal portion 28. When assembling the proximal assembly 13, the distal portion 28 of the dispense member 10 is positioned in the proximal housing 8. After that, the fixation part 15 is pushed over the proximal portion 27 of the dispense member 10 until the fixation part 15 engages with the proximal housing 8. In particular, the fixation part 15 may comprise coupling protrusions (not depicted) at an inner surface engaging in coupling holes 32, 33 at the proximal end of the proximal housing 8. This engagement prevents the proximal assembly 13 from being disassembled after assembly.

Regarding the components of the distal assembly 14, the needle guard 12 has a proximal portion 34, a middle portion 80 and a distal portion 35. A flange 36 extends radially outwards and is located between the proximal portion 34 and the middle portion 80. The proximal portion 34 comprises two racks 37, 38. The racks 37, 38 are two portions extending from the flange 36 in a proximal direction. The racks 37, 38 may have the shapes of rail-shaped extensions.

In specific embodiments, the racks 37, 38 interact with the flexible arms 29, 30 of the dispense member 10 in order to unlock the dispense member 10 and enable a dispense operation. In this case, the racks 37, 38 provide release devices 102, 103. Additionally or alternatively, the racks 37, 38 may be involved in locking the needle guard 12 after use. In this case, the racks 37, 38 provide first lock-out devices 104, 105.

The distal housing 9 comprises two lead-throughs 39, 40 for accommodating the racks 37, 38 and allowing a movement of the needle guard 12 in a proximal direction for exposing the needle. The lead-throughs 39, 40 are open toward a proximal and distal direction such that the racks 37, 38 are enabled to protrude out of the lead-throughs 39, 40 in both directions.

The distal housing 9 comprises a shoulder 41 and openings 42, 43 above the shoulder 41 for accommodating retention protrusions 44, 45 of the needle guard 12. The retention protrusions 44, 45 cooperate with the shoulder 41 to prevent the needle guard 12 from slipping out of the distal end of the distal housing 9.

Furthermore, an inner face at the shoulder 41 interacts with the flange 36 of the needle guard 12, when the needle guard 12 is moved in a proximal direction for exposing the needle 21 and, thus, limits movement of the needle guard 12 in the proximal direction.

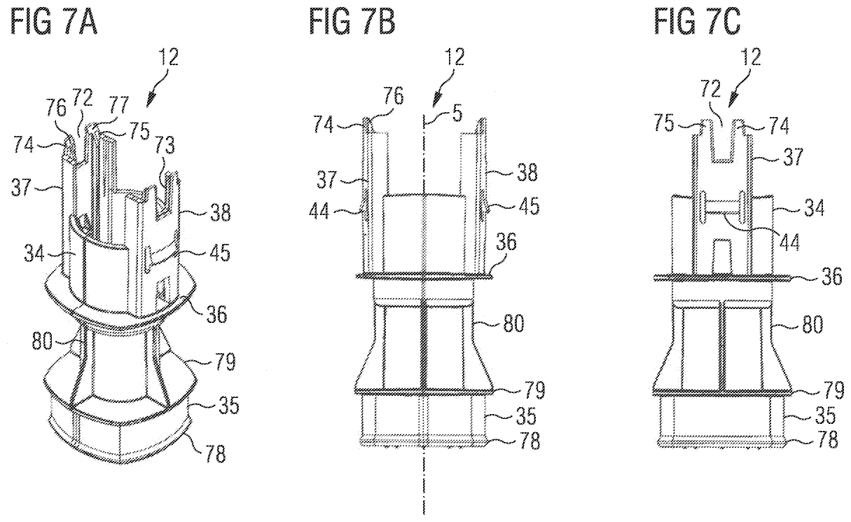

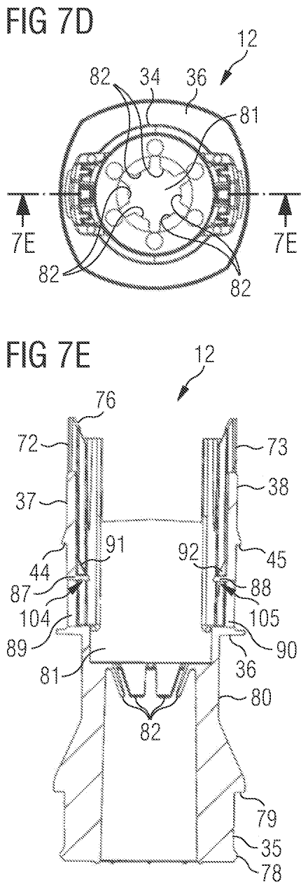

As can be further seen, the cap grip 16 has the shape of a ring. When forming the distal assembly 14, the cap grip 16 is fixed to the protective cap 11, by a snap-in connection, for example. The cap grip 16 serves to grip the needle cap 22 when the syringe 2 is placed in the distal assembly 14. When the protective cap 11 is removed, the protective cap 11 together with the cap grip 16 pulls the needle cap 22 off the syringe 2 and exposes the needle 21. A specific embodiment of the cap grip 16 is described in connection with FIG. 7D.

For assembling the distal assembly 14, the cap grip 16 is fixed to the protective cap 11 and then the protective cap 11 is attached to the distal portion 35 of the needle guard 12. After that, the needle guard 12 is pushed through the distal end of the distal housing 9 in the proximal direction, until the retention protrusions 44, 45 protrude out of the openings 42, 43 and are located directly above the shoulder 41. In this position, a proximal surface of the protective cap 11 abuts a distal surface of the distal housing 9 such that the components of the distal assembly 14 are locked in their positions. In particular, any movement of the needle guard 12 relative to the distal housing 9 is disabled.

In the assembled state, the components of the proximal assembly 13 are locked such that relative movements of the components are prevented in the proximal assembly 13. Additionally, the components of the distal assembly 14 are locked such that relative movements of the components are prevented in the distal assembly 14. This simplifies the handling of the device 1, in particular the assembly of the proximal assembly 13 with the syringe 2 and with the distal assembly 14, because no movable components are involved.

FIGS. 4A to 4C show the proximal assembly 13 in a side view, in a top view and in a longitudinal-sectional view, respectively. The line of intersection for the longitudinal sectional view is indicated in FIG. 4B by a line marked with “4C-4C”.

As can be seen in FIG. 4C, the proximal housing 8 has an inner surface 46 and retention protrusions 47, 48 protruding from the inner surface 46. The retention protrusions 47, 48 serve as an abutment face for distal end faces of the flexible arms 29, 30 of the dispense member 10 during formation of the proximal assembly 13 and limit how far the dispense member 10 can be pushed in a distal direction.

The retention protrusions 47, 48 serve as second locking devices 85, 86 for interaction with the first locking devices 83, 84 of the dispense member 10, to lock the dispense member 10 to the proximal housing 8. In particular, the interaction of the retention protrusions 47, 48 with the flexible arms 29, 30 prevent a distal movement of the dispense member 10 relative to the proximal housing 8, both before and after coupling of the proximal assembly 13 with the distal assembly 14.

In addition to that, a movement of the dispense member 10 in the proximal direction is prevented by an interaction of the flange 31 of the dispense member 10 with the fixation part 15.

The proximal housing 8 further comprises an annular retention ledge 49 defining first and second armholes 50, 51. The armholes 50, 51 are positioned on opposite sides of the proximal housing 8. Both armholes 50, 51 extend parallel to the centerline 5 and are open to both a proximal face and a distal face of the annular retention ledge 49. The retention protrusions 47, 48 are positioned in the first and second armholes 50, 51, respectively. The retention protrusions 47, 48 do not extend all the way across the armholes 50, 51 but leave a gap of sufficient width to allow the flexible arms 29, 30 of the dispense member 10 to pass through the armholes 50, 51 during a dispense operation, as described in detail in connection with FIG. 9B, for example.

FIG. 4C also shows the coupling protrusion 19 that interacts with the coupling portion 17 of the distal housing 9 and is guided in the guiding groove 18 for coupling the proximal housing assembly 13 to the distal housing assembly 14. The coupling protrusion 19 projects from the inner surface 46 of the proximal housing 8 and is positioned between the retention ledge 49 and the distal end of the proximal housing 8.

The dispense member 10 comprises a post 52, in particular a tubular post, extending from the proximal end 6 of the dispense member 10 in the distal direction. The post 52 directly interacts with the plunger 26 of the syringe 2 to push the plunger 26 in a distal direction during a dose dispense operation. Accordingly, the post 52 directly transmits the pushing force exerted by a user on the dispense surface to the plunger 26.

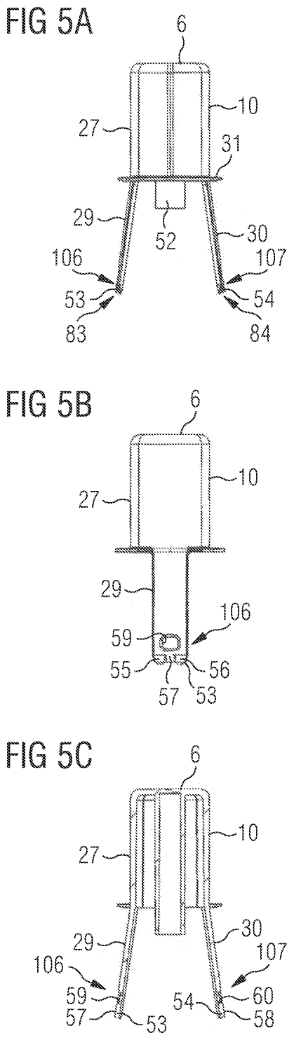

FIGS. 5A to 5C show the dispense member 10 in a side view, a side view rotated by an angle of 90° compared to FIG. 5A and a longitudinal-sectional view, respectively.

As can be seen in FIGS. 5A and 5C, the post 52 protrudes out of the proximal portion 27.

The first and second flexible arms 29, 30 are positioned on opposite sides of the dispense member 10 and extend from the distal end of the proximal portion 27 in a distal direction.

The flexible arms 29, 30 are flared such that the distance between each flexible arm 29, 30 and the centerline 5 increases in the distal direction. The flexible arms 29, 30 are resilient. When tips 53, 54 of the flexible arms 29, 30, i.e., the distal ends of the flexible arms 29, 30, are pressed towards the centerline 5, a resilient counterforce is provided by the flexible arms 29, 30.

When the flexible arms 29, 30 are positioned in the proximal housing 8 after forming the proximal assembly 13 and before using the device 1, the tips 53, 54 are pressed slightly towards the centerline 5 by an inner surface 46 of the proximal housing 8 such that the flexible arms 29, 30 are resiliently biased and a counterforce is exerted by the tips 53, 54 on the inner surface 46. This resilient force prevents the flexible arms 29, 30 from moving towards the centerline 5 and from disengaging from the retention protrusions 47, 48. Accordingly, the flexible arms 29, 30 serve as first locking devices 83, 84 for locking the dispense member 10 before proximal movement of the needle guard 12.

As can be seen in FIG. 5B, the tip 53 of the flexible arm 29 has beveled surfaces 55, 56. The beveled surfaces 55, 56 face the inner surface 46 of the proximal housing 8. Between the beveled surfaces 55, 56 of the tip 53, a protruding portion 57 is located, the protruding portions 57 having a thickness larger than the ends of the beveled surfaces 55, 56. Due to the larger thickness, the protruding portion 57 abuts on the inner surface 46 such that a gap is provided between the beveled surfaces 55, 56 and the inner surface 46. The beveled surfaces 55, 56 and the protruding portion 57 interacts with the rack 37 of the needle guard 12 for unlocking the dispense member 10 to enable a movement in a distal direction. The second flexible arm 30 has a corresponding shape and a corresponding interaction of a protruding portion 58 with the second rack 38. The details of the mechanism are described in connection with FIG. 9B, for example.

As can be further seen in FIGS. 5A to 5C, each of the flexible arms 29, 30 has an opening 59, 60 near the distal end. These openings 59, 60 serve as first lock-out devices 104, 105 for locking the dispense member 10 with the needle guard 12 after a dispense operation, as is described in connection with FIG. 9C, for example.

FIGS. 6A to 6E show the distal housing 9 in a perspective view, a side view, a rotated side view, a top view and a longitudinal-sectional view, respectively. The line of intersection for the longitudinal-sectional view is indicated in FIG. 6D by a line marked with “6E-6E”.

The distal housing 9 has a proximal portion 61 and a distal portion 62. The perimeter of the distal portion 62 is larger than the perimeter of the proximal portion 61. The shoulder 41, which prevents slipping of the needle guard 12 out of the distal housing 9, is formed between the proximal portion 61 and the distal portion 62. The distal portion 62 has an outer perimeter that has the same shape and size as the outer perimeter of the proximal housing 8. The outer perimeter of the proximal portion 61 has the same shape, but is slightly smaller than the inner surface 46 of the proximal housing 8 such that, in an assembled state, the proximal portion 61 is accommodated in the proximal housing 8.

In the following, a specific embodiment of the coupling mechanism for coupling the proximal assembly 13 to the distal assembly 14 is described in connection with FIGS. 6A and 6B. In other embodiments, the coupling mechanism may be different.

The coupling portion 17 comprises the guiding groove 18 that forms a track for guiding the coupling protrusion 19 of the proximal housing 8. The guiding groove 18 has a first portion 63 that receives the coupling protrusion 19 and is open towards the proximal edge of the distal housing 9. The first portion 63 has a larger width at its proximal end in order to facilitate leading the coupling protrusion 19 in the groove 18.

The groove 18 has a second portion 64 that is at an angle from the first portion 63. An end 65 of the groove 18 is closed and forms a hard stop for the coupling protrusion 19 and hence stops rotation of the proximal housing 8 relative to the distal housing 9.

A snap hole 66 is formed through the wall of the distal housing 9 and extends from the surface of the groove 18 to the inner surface of the distal housing 9. A latch 67 is located at the wall of the distal housing 9 and extends into the snap hole 66. The latch 67 is resilient and can flex into the inner volume of the distal housing 9 toward the centerline 5. The end portion of the latch 67 has a retention surface 68 that ramps radially outward away from the centerline 5 and toward the outer perimeter of the distal housing 9.

When the proximal assembly 13 and the distal assembly 14 are connected to place the device 1 into an operational state, the coupling protrusion 19 is positioned into and moved through the groove 18 until it engages and moves over the retention surface 68. As the coupling protrusion 19 moves over the retention surface 68, the latch 67 flexes and moves toward the inner chamber of the distal housing 9. When the coupling protrusion 19 passes the retention surface 68, the flange 67 snaps, or otherwise returns to its resting position and the coupling protrusion 19 is held securely between the retention surface 68 and the end 65 defining a hard stop surface of the groove 18.

The snapping action of the latch 67 when the coupling protrusion 19 moves over the retention surface 68 may provide confirmation to a user that the proximal assembly 13 has been successfully attached to the distal assembly 14 such that the device 1 is in an operational state.

The retention surface 68 may have an increased thickness near the center of the retention surface 68 such that the retention surface 68 ramps up and down so that the coupling protrusion 19 may be enabled to move over the retention surface 68 in both directions, enabling a user to both attach and detach the proximal assembly 13 to and from the distal assembly 14. According to a different embodiment, the coupling mechanism may be configured such that a detachment of the proximal assembly 13 from the distal assembly 14 is disabled.

As can be also seen in FIGS. 6A to 6E, the cradle 25 is formed by a shallow wall that projects up at a proximal end of the distal housing 9. The perimeter of the shallow wall has a shape and size similar to the shape and size of the fitting 24 on the syringe flange 23. When the syringe 2 is inserted into the distal assembly 9, the syringe fitting 24 and flange 23 fit within the cradle 25. When the proximal assembly 13 is connected to the distal assembly 14 the depth of the space between the proximal surface of the distal housing 9 and the distal surface of the annular retention ledge 49 in the proximal housing 8 is slightly smaller than the thickness of the syringe fitting 24. Thereby, the syringe fitting 24 and flange 23 are pinched between the proximal housing 8 and distal housing 9 and cannot move axially in an assembled state of the device 1. The cradle 25 also prevents the syringe 12 from rotating.

As can best be seen in FIG. 6E, the cradle 25 has an opening 69 in its bottom surface leading to a tubular post 70 in which the syringe barrel 20 is accommodated and confined in its position relative to the distal housing 9. The tubular post 70 has a smaller outer perimeter than the perimeter of an inner surface 71 of an outer wall of the distal housing 9. The gap between the tubular post 70 and the inner surface 71 define the lead-throughs 39, 40 accommodating the racks 37, 38 of the needle guard 12.

FIGS. 7A to 7E show the needle guard 12 in a perspective view, a side view, a rotated side view, a top view and a longitudinal-sectional view, respectively. The line of intersection for the longitudinal-sectional view is indicated in FIG. 7D by a line marked with “7E-7E”.

The two racks 37, 38 are located on opposite sides of the needle guard 12 in the proximal portion 34 of the needle guard 12. The racks 37, 38 extend parallel to the centerline 5. The racks 37, 38 are rigid, i.e. non-flexible. The proximal edge of each rack 37, 38 defines a slot 72, 73 open to the proximal edge. The slots 72, 73 receive the retention protrusions 47, 48 at the inner surface 46 of the proximal housing 8 when releasing the locking of the dispense member 10.

The slot 72 is confined by two proximal tip portions 74, 75, each tip portion 74, 75 having a beveled surface 76, 77 at an inwardly facing surface of the needle guard 12. The beveled surfaces 76, 77 are sized, shape and arranged to slide in between the beveled surfaces 55, 56 of the tip 53 of the flexible arm 29 of the dispense member (see FIG. 5B) and the inner surface 46 of the proximal housing 8 when the needle guard 12 moves in a proximal direction to unlock the dispense member 10. The second rack 38 has a corresponding interaction with the second flexible arm 30.

As explained above, the needle guard 12 has retention protrusions 44, 45 interacting with the shoulder 41 in the proximal housing 9 to prevent slipping of the needle guard 12 out of the housing 9. Furthermore, the flange 36 has an outer perimeter smaller than the perimeter of the inner surface of the distal portion 62 of the distal housing 9 and is larger than the perimeter of the inner surface of the proximal portion 61 of the distal housing 9. This enables the needle guard 12 to move along the centerline 5 in the distal portion 61 of the distal housing 9 until the flange 36 abuts a part of an inner wall at the shoulder 41.

At the bottom edge of the distal portion 35, the needle guard 12 has a cap retention flange 78 extending radially outwards. The flange 78 serves to hold the protective cap 11 in place when the protective cap 11 is attached to the distal assembly 14 prior to use.

The needle guard 12 further has an annular flange 79 between the distal portion 35 and the middle portion 80. The annular flange 79 has an outer perimeter adapted to the perimeter of an inner surface of the distal housing 9 such that the needle guard 12 is securely guided along the inner surface of the distal housing 9.

As can be seen in FIG. 7D, the needle guard 12 defines a center channel 81 extending all the way through the needle guard 12. The center channel 81 has a perimeter larger than the outer perimeter of the syringe barrel 20. A plurality of projections 82 extend in the center channel 81 towards the centerline 5. Each of the projections 82 has a tip, and the tips of the projections 82 define a cross-sectional area that is orthogonal to the centerline 5 and that has a perimeter slightly smaller than the outer diameter of the syringe barrel 20. When placing the syringe 2 in the distal assembly 14, the barrel 20 engages the projections 82 as it passes through the center channel 81. The projections 82 center the barrel 20 and provide a slight amount of friction between the projections 82 and the barrel 20. Thereby, the projections 82 prevent the barrel 20 of the syringe 2 from moving laterally to the centerline 5 and still permit the needle shroud 12 to slide proximally along the length of the barrel 20.

As can be seen in the longitudinal-sectional view shown in FIG. 7E, the needle guard 12 comprises first lock-out devices 104, 105 located at a distal portion of the racks 37, 38. The lock-out devices 104, 105 interact with the second lock-out devices 106, 107 of the dispense member 10 to lock the needle guard 12 with the dispense member 10 after the full dose of medicament has been dispensed. Thereby, any relative movements of the dispense member 10 and the needle guard 12 are prevented. The locking is permanent and, thus, cannot be released by a user.

The first lock-out devices 104, 105 comprise lock-out protrusions 87, 88 protruding inwardly from an inner wall of the needle guard 12. In addition to that, the first lock-out devices 104, 105 comprise lock-out openings 89, 90 leading through an inner wall of the needle guard 12. The lock-out protrusions 87, 88 are sized to fit into the first and second armholes 50, 51 at the distal ends of the flexible arms 29, 30 of the dispense member 10. The lock-out protrusions 87, 88 comprise ramped upper surfaces 91, 92 for pressing the tips 53, 54 of the flexible arms 29, 30 inwardly when the needle guard 12 moves in a distal direction and when the beveled surfaces 55, 56 of the tips 53, 54 slide along the ramped upper surfaces 91, 92.

In an alternative embodiment, lock-out may be provided entirely by a friction force between the flexible arms 29, 30 and the racks 37, 38. In this case, the lock-out openings 89, 90 and lock-out protrusions 87, 88 may not be present.

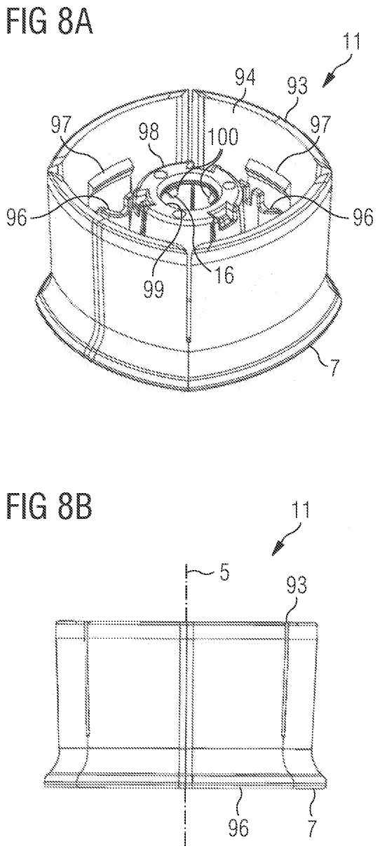

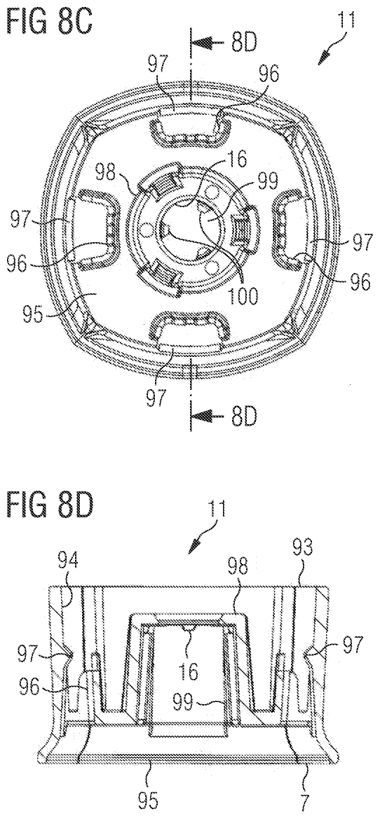

FIGS. 8A to 8D show the protective cap 11 in a perspective view, a side view, a top view and a longitudinal-sectional view, respectively. The line of intersection for the longitudinal-sectional view is indicated in FIG. 8C by a line marked with “8D-8D”.

The protective cap 11 has a proximal end 93 and a distal end 7, and defines an inner volume 94. The proximal end 93 is open and sized to receive the distal portion 35 of the needle guard 12. The distal end 7 is closed and has a bottom wall 96 that is orthogonal to the centerline 5. A plurality of retention members 96 is positioned in the inner volume 94 and extend upwards from the bottom wall 96. Near the proximal ends of the retention members 96 cap retention ridges 97 extend inwards toward the centerline 5. The radial distance from the centerline 5 to each of the cap retention ridges 97 is slightly smaller than the radius of the cap retention flange 78 of the needle guard 12. Thus, when the device 1 is in its assembled pre-use configuration, the cap retentions ridges 97 snap over the cap retention flange 78 and hold the protective cap 11 in place on the distal portion 35 of the needle guard 12.

The protective cap 11 can be removed from the needle guard 12 by pulling the protective cap 11 in a distal direction so that the cap retention ridges 97 snap over the cap retention flange 78. The cap retention ridges 97 are formed with a resilient material that enables them to flex at least slightly to provide the snap mechanism allowing the protective cap 11 to be attached to and removed from the needle guard 12.

A tubular post 98 extends upwards from the bottom of the protective cap 11 and into the inner volume 94. The tubular post 98 is open at its proximal end. The tubular post 98 defines a volume 99 sized to receive the needle cap 22. The needle cap grip 16 is attached to the tubular post 98. The needle cap grip 16 and the tubular post 98 are concentric. The tubular post 98 has an inner diameter that is larger than the needle cap 22. A plurality of teeth 100 extend from the inner edge of the needle cap grip 16, and have an end portion. The radial position between the centerline 5 and the end portion of the teeth 100 is smaller than the outer diameter of the needle cap 22. When the syringe 2 is inserted into the distal assembly 14, the teeth 100 securely grip the needle cap 22. When the protective cap 11 is removed from the needle guard 12, the protective cap 11 and needle cap grip 16 pull the needle cap 22 off the syringe 2 and expose the needle 21.

The operation of the assistive device 1 comprising the syringe 2 is described now in detail in connection with FIGS. 9A, 9B and 9C.

The assistive device 1 and therewith, the medication delivery device 4, has several states. These states may all occur after distribution to a user.

A first state is the pre-assembled state as shown in FIG. 2. In the pre-assembled state, the proximal assembly 13, the distal assembly 14 and the syringe 2 are not yet assembled with each other.

A further state is an operational state, in which the device 1 is assembled with the syringe 2 and wherein a dispense operation has not started or a dose has not been fully dispensed. The operational state is depicted in FIGS. 9A and 9B, wherein FIG. 9A shows the device 1 before exposing the needle 21 and FIG. 9B shows the device 1 after exposing the needle 21 and before dispensing the medicament.

A further state is a locked-out state, in which the dispense member 10 has been fully depressed such that the dose has been fully dispensed and the device 1 is locked-out to prevent a re-use of the device 1. The locked-out state is shown in FIG. 9C.

Returning to the pre-assembled state as shown in FIG. 2, a user assembles the components by placing the syringe 2 in the distal assembly 14. In particular, the syringe 2 is inserted into the opening 69 (see FIG. 6D) of the distal housing 9 and the center channel 81 (see FIG. 7D) of the needle guard 12 until the syringe flange 23 and the syringe fitting 24 are seated in the cradle 25 and rest against the proximal surface of the distal housing 9. In this position, the needle cap 22 is inserted into the needle cap grip 16 in the protective cap 11 so that the teeth 100 grip the outer surface of the needle cap 22.

The user then attaches the proximal assembly 13 to the distal assembly 14 to place the device 1 in the assembled state. To attach the assemblies 13, 14, the user inserts the coupling protrusion 19 all the way through the first portion 63 of the guiding groove 18. In this position, the plunger 26 of the syringe 2 is positioned in the dispense member 10. The user then twists the proximal assembly 13 relative to the distal assembly 14 so that the coupling protrusion 19 travels through the second portion 64 of the guiding groove 18 and clicks over the retention surface 68 of the latch 67.

The device 1 is now in the operational state and ready to use. However, as long as the protective cap 11 is attached, a movement of the needle guard 12, and thereby also of the dispense member 10 is prevented. In particular, the proximal end 93 of the protective cap 11 abuts a distal end of the distal housing 9 and, thereby, blocks the protective cap 11 and the needle guard 12 from moving in a proximal direction.

Furthermore, the dispense member 10 is locked in its position due to the interaction of the first locking devices 83, 84, in particular the tips 53, 54 of the flexible arms 29, 30, with the second locking devices 85, 86, in particular, the retention protrusions 47, 48.

To start an operation, the user pulls the protective cap 11 off the distal portion 35 of the needle guard 12.

FIG. 9A shows the device 1 in the state after removal of the protective cap 11 and, thereby, also of the needle cap 22. The tip of the needle 21 is covered by the distal portion 35 of the needle guard 12 and is not exposed. This state is an operational state ready to pierce a skin by the needle 21. The needle guard 12 is movable in a proximal direction.

In this state, the dispense member 10 is still locked, i.e. unable to move in a distal direction. The tubular post 52 of the dispense member 10 is located above the plunger 26. A gap may exist between the tubular post 52 and the plunger 26.

In a second step of operation, the needle guard 12 is moved in a proximal direction and the device 1 is thereby brought in an operational state ready to dispense the medicament as shown in FIG. 9B. This means that the movement of the dispense member 10 is now enabled without that any further unlocking action is required.

In particular, starting from the state shown in FIG. 9A, the user presses a distal surface 101 of the needle guard 12 against his/her skin at the injection site and presses the device 1, in particular the assembled housings 8, 9 towards the skin. As the housings 8, 9 move towards the skin, the needle guard 12 is pushed in a proximal direction relative to the housings 8, 9. The distal portion 35 of the needle guard 12 slides into the distal housing 9 until the flange 36 of the needle guard 12 abuts with a distal surface at the shoulder 41. The distal surface 101 of the needle guard 12 may now be flush with the distal end of the distal housing 9. The needle 21 is exposed and pierces the skin.

As the needle guard 12 moves in the proximal direction, the racks 37, 38 slide through the lead-throughs 39, 40 further in the proximal housing 8. The beveled surfaces 76, 77 of the tip 74, 75 of the rack 37 slide on the beveled surfaces 55, 56 of the tip 53 of the flexible arm 29. Corresponding interactions occur between the second rack 38 and second flexible arm 30. In this action, the first and second racks 37, 38 slide between the first and second flexible arms 29, 30 of the dispense member 10 and the inner surface 46 of the proximal housing 8. Thereby, the tips 53, 54 of the flexible arms 29, 30 are moved inwardly and are separated from the inner surface 46 of the proximal housing 8. The tips 53, 54 are in a radial position inwardly from the retention protrusions 47, 48 and, thus, off from the retention protrusions 47, 48.

The tip portions 74, 75 of the rack 37 that defines the slot 72 move along either side of the retention protrusion 47 so that the retention protrusion 47 is now positioned in the slots 72 of the rack 37 respectively. Corresponding interactions occur between the second rack 38 and second retention protrusion 48. The dispense member 10 is now free to move and to be depressed into the proximal housing 8. Accordingly, the racks 37, 38 provide release devices 102, 103 for releasing the locking of the dispense member 10 with the proximal housing 8.

In a third step of operation, the medicament is dispensed by moving the dispense member 10 in the distal direction. When the dispense member 10 is fully depressed, the device 1 acquires a locked-out state as shown in FIG. 9C.

To inject medication, the dispense member 10 is moved in a distal direction, e.g., by placing the thumb on the proximal end 6 of the dispense member and pushing downwards. As the dispense member 10 is depressed, it moves through the proximal housing 8 and presses the syringe plunger 26 into the barrel 20. Accordingly, the dispense member 10 directly acts on the plunger 26. Thus, the dispense member 10 and the plunger 26 carry out identical movements during dispense of the medication. The plunger 26 moves forwards in the barrel 20 and pushes the medicament out of the needle 21.

As the dispense member 10 moves, the first and second flexible arms 29, 30 slide along the surfaces of the racks 37, 38, respectively. The flexible arms 29, 30 are pretensioned and provide a resilient force on the racks 37, 38. Due to the resilient force, the dispense member 10 remains in its axial position when inadvertently released during a dose dispense operation.

At the end of the dispense operation, the first lock-out devices 104, 105 of the needle guard 12 interlock with the second lock-out devices 106, 107 of the dispense member 10. In particular, the beveled surfaces 55, 56 of the tip 53 of the flexible arm 29 slide along ramped upper surfaces of the lock-out protrusion 87 of the needle guard 12. Corresponding interactions occur between the second flexible arm 30 and second lock-out protrusion 88. Thereby, the tips 53, 54 of the flexible arms 29, 30 move inwardly, pass the lock-out protrusions 87, 88 and snap outwards under the lock-out protrusions 87, 88. The lock-out protrusions 87, 88 are now located in the openings 59, 60 in the distal portions of the flexible arms 29, 30.

This action interlocks the dispense member 10 and the needle guard 12 and holds them in their used final positions. In particular, the dispense member 10 can no longer slide proximally back out of the proximal housing 8 and the needle guard 12 can no longer distally slide out of the distal housing 9. Accordingly, the dispense member 10 remains permanently in a fully depressed position and the needle guard 12 remains permanently in a fully retracted position. Thus, also the needle 21 remains permanently exposed after usage. This clearly signals to a user that the device 1 has been used for dispensing the medicament and should be discarded.

In an alternative embodiment, the lock-out mechanism is provided by the friction force between the flexible arms 29, 30 and the racks 37, 38. When the dispense member 10 is fully depressed, a manual extension of the dispense member 10 or the needle guard 12 may be impractical for a user. The same applies to a manual extension of the needle guard 12.

The interlocking of the dispense member 10 and the needle guard 25 also keeps the proximal assembly 13 and the distal assembly 14 from being detached and prevents the syringe 2 from being removed from the device 1 and being used again. This may increase the safety of the device 1.

### REFERENCE NUMERALS

1 assistive device
2 syringe
3 housing
4 medication delivery device
5 centerline
6 proximal end
7 distal end
8 proximal housing
9 distal housing
10 dispense member
11 protective cap
12 needle guard
13 proximal assembly
14 distal assembly
15 fixation part
16 cap grip
17 coupling portion
18 guiding groove
19 coupling protrusion
20 barrel
21 needle
22 needle cap
23 flange of syringe
24 fitting
25 cradle
26 plunger
27 proximal portion of dispense member
28 distal portion of dispense member
29 first flexible arm
30 second flexible arm
31 flange of dispense member
32 first coupling hole
33 second coupling hole
34 proximal portion of needle guard
35 distal portion of needle guard
36 flange of needle guard
37 first rack
38 second rack
39 first lead-through
40 second lead-through
41 shoulder
42 first opening
43 second opening
44 first retention protrusion
45 second retention protrusion
46 inner surface of proximal housing
47 first retention protrusion
48 second retention protrusion
49 annular retention ledge
50 first armhole
51 second armhole
52 tubular post
53 first tip
54 second tip
55 first beveled surface of flexible arm
56 second beveled surface of flexible arm
57 protruding portion
58 protruding portion
59 first opening
60 second opening
61 proximal portion of distal housing
62 distal portion of distal housing
63 first portion of groove
64 second portion of groove
65 end of groove
66 snap hole
67 latch
68 retention surface
69 opening
70 tubular post of distal housing
71 inner surface of outer wall of distal housing
72 slot
73 slot
74 first tip portion
75 second tip portion
76 first beveled surface
77 second beveled surface
78 cap retention flange of needle guard
79 annular flange of needle guard
80 middle portion of needle guard
81 center channel
82 plurality of projections
83 first locking device of dispense member
84 first locking device of dispense member
85 second locking device of housing
86 second locking device of housing
87 lock-out protrusion
88 lock-out protrusion
89 lock-out opening
90 lock-out opening
91 ramped upper surface
92 ramped upper surface
93 proximal end of protective cap
94 inner volume
95 bottom wall
96 retention members
97 cap retention ridges
98 tubular post of protective cap
99 inner volume of tubular post
100 plurality of teeth
101 distal surface of needle guard 12
102 first release device
103 second release device
104 first lock-out device of needle guard
105 first lock-out device of needle guard
106 second lock-out device of dispense member
107 second lock-out device of dispense member

1. An assistive device for use with a syringe,
the device comprising a housing for accommodating the syringe,
a dispense member to be moved in a distal direction for dispensing a medicament from the syringe,
a needle guard having a distal position for covering a needle of the syringe before operation of the device, wherein the needle guard is moveable from the distal position to a proximal position to expose the needle,
wherein the dispense member comprises a first locking device and the housing comprises a second locking device for locking the dispense member to the housing when the needle guard in is the distal position, such that a movement of the dispense member in the distal direction is prevented.

2. The assistive device of claim 1,
wherein the needle guard comprises a release device for unlocking the dispense member when the needle guard moves in its proximal position.

3. The assistive device of claim 1,
wherein the dispense member comprises a flexible arm providing the first locking device.

4. The assistive device of claim 1,
wherein the second locking device comprises a protrusion at an inner surface of the housing.

5. The assistive device of claim 1,
wherein the first locking device is configured to be deflected inwardly by an interaction with the release device.

6. The assistive device of claim 1,
wherein the release device is configured to slide between the housing and the first locking device when the needle guard moves in a proximal direction.

7. The assistive device of claim 3,
wherein the flexible arm is configured to be pretensioned and thereby hold the dispense member in its position when the dispense member is released during a dispense operation.

8. The assistive device of claim 1,
wherein the housing comprises a proximal housing and a distal housing to be connected to each other for assembly of the device,
wherein the proximal housing comprises the second locking member and the dispense member is locked with the proximal housing prior to assembly of the device.

9. The assistive device of claim 1,
being a single-use device such that only a single dispense operation is enabled.

10. The assistive device of claim 1,
configured such that when the needle guard is in the proximal position and the dispense member is in a distal position, the needle guard interacts with the dispense member such that the dispense member and/or the needle guard is held in its axial position.

11. The assistive device of claim 10,
wherein the needle guard comprises a first lock-out device and the dispense member comprises a second lock-out device engaging each other for locking the needle guard to the dispense member after a dose dispense operation.

12. The assistive device of claim 1, configured such that the needle is permanently visible after dispense of the medication and release from a user's skin.

13. The assistive device of claim 1,
wherein the housing comprise a proximal housing and a distal housing to be connected to each other for assembly of the device,
wherein a disconnection of the proximal housing from the distal housing is disabled after dispense of the medication.

14. The assistive device of claim 1,
wherein the syringe to be used with the device comprises a needle and a plunger protruding out of the barrel.

15. A kit comprising the assistive device of claim 1 and comprising a syringe accommodated in the assistive device, wherein the syringe is prefilled with a pharmaceutical preparation comprising fremanezumab.
